(19) United States
(12) Patent Application Publication
(10) Pub. No.: US 2021/0051953 A1
(43) Pub. Date: Feb. 25, 2021

(54) CLEANING AND/OR DISINFECTING COMPOSITIONS

(71) Applicant: LEGIOGUARD PTY LTD, Alexandria (AU)

(72) Inventors: Andrew Scott Collins, Warradale (AU); Scott Kennedy Goulter, Robina (AU)

(73) Assignee: LEGIOGUARD PTY LTD, Alexandria (AU)

(21) Appl. No.: 16/963,049

(22) PCT Filed: Jan. 18, 2019
(86) PCT No.: PCT/AU2019/000008

§ 371 Date: Jul. 17, 2020

(30) Foreign Application Priority Data
Jan. 19, 2018 (AU) 2018900175
Jun. 28, 2018 (AU) 2018902326

(51) Int. Cl. A01N 37/02; C11D 3/20; C11D 3/48; C11D 3/04; A01N 59/08; A01N 43/08; C11D 11/00; A61L 2/18

(52) U.S. Cl. CPC A01N 37/02; C11D 3/2079; C11D 3/48; C11D 3/046; A61L 2202/17; A01N 43/08; C11D 3/2096; C11D 11/0041; A61L 2/18; A01N 59/08

(57) ABSTRACT

A cleaning and/or disinfecting composition comprising: at least one carboxylic acid; at least one alkali metal halide salt; and at least one antibacterial compound is disclosed. The composition can be used to clean and/or disinfect a surface or water system.

### PRIORITY DOCUMENTS

The present application claims priority from Australian Provisional Patent Application No. 2018900175 titled “CLEANING AND/OR DISINFECTING COMPOSITIONS” filed on 19 Jan. 2018 and Australian Provisional Patent Application No. 2018902326 titled “CLEANING AND/OR DISINFECTING COMPOSITIONS II” filed on 28 Jun. 2018, the contents of which are hereby incorporated by reference in their entirety.

### TECHNICAL FIELD

The present disclosure relates to cleaning and/or disinfecting compositions. In a particular form the present disclosure relates to cleaning and/or disinfecting compositions for cleaning and/or disinfecting water supply lines, containers, cooling towers and similar wettable surfaces.

### BACKGROUND

In many settings, organic and inorganic deposits accumulate on the wettable surfaces of water distribution supply lines, containers and other surfaces. Organic deposits of yeast, bacteria, and other microorganisms form heterotroph and polysaccharide layers or “biofilms” that have been shown to harbour and protect pathogenic or otherwise troublesome bacteria, viruses, algae, algal toxins, fungi, proteobacteria, protozoa and invertebrates. Microorganisms within biofilms have been shown to periodically slough off and enter the flowing water, thereby contaminating other systems and exposing consumers to biological hazards from water distribution supply lines. If industrial water systems are not cleaned regularly they can become fouled and this has a negative impact on the performance of a system as mineral scale builds up on the surface of the water lines.

Evaporative cooling systems are particularly prone to fouling through air-borne contaminants, water-borne contaminants, and stagnation. There are serious health risks associated with microbiological contamination which can establish microbial communities forming biofilms on wettable surfaces of a cooling tower. These biofilms are complex communities offering nutrients and protection for growth further enhancing scale, corrosion and reduction in efficiency. They also can provide a source for legionella and other water-borne pathogens to proliferate.

Similarly, water systems within hospitals, healthcare and aged-care facilities can become fouled through water-borne contaminants and/or poor design. There are serious health risks associated with microbiological contamination in these systems.

Similarly, microbial infection of non-wettable surfaces on medical devices, medical catheters, stents, hospital equipment etc also presents serious health risks.

Typically, chemical cleaners or sanitisers rely on hyperhalogenation or caustic solutions to reduce or eliminate bacterial growths or infections. However, relatively high concentrations are generally required (e.g. up to 20 ppm for chlorine based chemicals) and, if not flushed correctly after cleaning, these chemicals have negative health implications. Caustic solutions are also dangerous to humans and require a certain degree of care when handling. Further, they are not entirely successful in removing biofilms.

There is thus a need to provide a safe and non-toxic alternative to clean and/or disinfect wettable and non-wettable surfaces in water systems, cooling systems, medical equipment, medical devices, and similar.

### SUMMARY

The present disclosure results from the inventor(s) research and subsequent development of a liquid non-toxic solution that can be used to effectively clean and disinfect a range of surfaces in industrial, scientific, professional and consumer markets.

In a first aspect, the present disclosure provides a cleaning and/or disinfecting composition comprising: at least one carboxylic acid; at least one alkali metal halide salt; and at least one antibacterial compound.

The composition of the first aspect may optionally comprise an aqueous or nonaqueous vehicle. The composition can be in the form of a solution, an emulsion, a dip, a spray, or similar.

In a second aspect, the present disclosure provides a method for cleaning and/or disinfecting a surface, the process comprising contacting the surface to be cleaned or disinfected with a composition of the first aspect for a temperature and a time that is sufficient to clean and/or disinfect the surface.

The surface to be cleaned and/or disinfected can be any surface, such as the surface of a water supply line, the surface of a container, the surface of a cooling tower, the surface of medical equipment, exposed surfaces in food processing plants, residential, hospital, restaurants, public facilities and the like, skin of animals and humans, contaminated open wounds and tissue, dermal wound sites and/or lesions of living organisms such as animals and humans.

In certain embodiments, the surface to be cleaned and/or disinfected is a surface contained in a water supply line, container, cooling tower or similar surface. Thus, in a third aspect, the present disclosure provides a method of cleaning and/or disinfecting a water system comprising the steps of: a) providing a water system; b) adding to the water of the water system a composition of the first aspect; c) allowing the water to circulate through the water system; and d) draining the water from the water system.

The composition comprises at least one carboxylic acid. The carboxylic acid may be a C₁ to C₆ carboxylic acid. In certain embodiments, the carboxylic acid is acetic acid.

The composition also comprises at least one alkali metal halide salt. The metal halide salt may be selected from the group consisting of sodium chloride, potassium chloride, lithium chloride, sodium bromide, potassium bromide, lithium bromide, potassium iodide, and calcium chloride. In certain embodiments, the alkali metal halide salt is sodium chloride.

The composition also comprises at least one antibacterial compound. In certain embodiments, the antibacterial compound is a bacterial quorum sensing inhibitory compound. In certain embodiments, the antibacterial compound is ascorbic acid and/or a mineral ascorbate. The mineral ascorbate may be selected from the group consisting of sodium ascorbate, calcium ascorbate, potassium ascorbate, and magnesium ascorbate. In certain embodiments, the antibacterial compound is sodium ascorbate. In certain other embodiments, the antibacterial compound is calcium ascorbate.

Optionally, the composition further comprises one or more additives or functional chemicals. Suitable additives or functional chemicals that can be used include dyes, perfumes, biocides, corrosion inhibitors, dispersants, surfactants, reducing agents and pH adjusters.

The composition can be used to clean and/or disinfect fluid conduits (e.g. pipes) and other wetted surfaces of water distribution, water recirculation, and/or water treatment systems. The composition can be also be used to clean and/or disinfect water system equipment, floors, walls, and drains. Furthermore, the composition may be used to treat skin infections caused by pseudomonas resistant to antibiotics.

### BRIEF DESCRIPTION OF THE FIGURE

Embodiments of the present disclosure will be discussed with reference to the accompanying FIGURE wherein:

FIG. 1 is a plot of heterotroph count on a cross linked polyethylene pipe before and after treatment with a composition of embodiments of the present disclosure (T=tempered water and C=cold water).

### DESCRIPTION OF EMBODIMENTS

The present disclosure provides a cleaning and/or disinfecting composition. The composition comprises: at least one carboxylic acid; at least one alkali metal halide salt; and at least one antibacterial compound.

The present composition is a synergistic combination of a carboxylic acid (such as acetic acid), a metal halide salt (such as sodium chloride), and an antibacterial compound (such as sodium ascorbate).

As used herein, the term “cleaning”, and related terms, means reducing the overall quantity of deposits. Also as used herein, the term “disinfecting”, and related terms, means killing microorganisms, including bacteria, virus, fungi and other micro-organisms. As used herein, the goal of disinfection is to cause an overall significant reduction in the number or viability of microorganisms within a system.

The composition comprises at least one carboxylic acid. The carboxylic acid may be a C₁ to C₆ carboxylic acid. For example, the carboxylic acid may be selected from the group consisting of formic acid, acetic acid, glycolic acid, oxalic acid, propionic acid, lactic acid, butyric acid, and citric acid. In certain embodiments, the carboxylic acid is a food grade carboxylic acid or a carboxylic acid that is safe for human consumption.

In certain embodiments, the carboxylic acid is acetic acid. It is known that certain concentrations of acetic acid are effective for inhibiting the growth of various microorganisms (J. D. Reid, Am. J. Hygiene 1932, 16, 540). The carboxylic acid component of the composition may be an aqueous solution of the carboxylic acid, such as vinegar (i.e. a 5%-20% acetic acid solution). The pH of the acetic acid solution may be from about 2 to about 3. Thus, the carboxylic acid component of the composition may be a carboxylic acid solution having a pH of from about 2 to about 3. The acetic acid content of the vinegar provides antibacterial and antifungal properties with broad spectrum of activity against Streptococci, Staphylococci, Pseudomonas, and other proteobacteria, bacteria and fungi. The acetic acid also decreases the pH of the aqueous solution and, hence, the composition as a whole.

Suitable carboxylic acids are commercial chemicals available from various chemical suppliers. These acids can be purchased in dry or in liquid form or in formulations that can be in dry or liquid form.

The amount of carboxylic acid in the composition will depend on the specific carboxylic acid used, and/or the pH of the carboxylic acid used in aqueous solution, and/or the type of water system to be cleaned and/or disinfected. The person skilled in the art can calculate how much carboxylic acid is required in order to achieve a desired pH. In certain embodiments, the carboxylic acid is present in an amount of from about 5% w/v to about 50% w/v, such as about 5% w/v, about 6% w/v, about 7% w/v, about 8% w/v, about 9% w/v, about 10% w/v, about 11% w/v, about 12% w/v, about 13% w/v, about 14% w/v, about 15% w/v, about 16% w/v, about 17% w/v, about 18% w/v, about 19% w/v, about 20% w/v, about 21% w/v, about 22% w/v, about 23% w/v, about 24% w/v, about 25% w/v, about 26% w/v, about 27% w/v, about 28% w/v, about 29% w/v, about 30% w/v, about 31% w/v, about 32% w/v, about 33% w/v, about 34% w/v, about 35% w/v, about 36% w/v, about 37% w/v, about 38% w/v, about 39% w/v, about 40% w/v, about 41% w/v, about 42% w/v, about 43% w/v, about 44% w/v, about 45% w/v, about 46% w/v, about 47% w/v, about 48% w/v, about 49% w/v, or about 50% w/v. In certain embodiments, the carboxylic acid is acetic acid and the concentration of acetic acid in the composition is from about 5% w/v to about 15% w/v, such as about 9.9% w/v. Note that the cleaning and/or disinfecting composition may be further diluted prior to use. For example, the cleaning and/or disinfecting composition may be diluted 1:1 with water prior to use. Unless otherwise specified, the concentrations referred to herein refer to the concentration in the cleaning and/or disinfecting composition prior to dilution.

The composition also comprises at least one alkali metal halide salt. The metal halide salt may be selected from the group consisting of sodium chloride (i.e. common salt), potassium chloride, lithium chloride, sodium bromide, potassium bromide, lithium bromide, potassium iodide, and calcium chloride. In certain embodiments, the alkali metal halide salt is sodium chloride. Sodium chloride has been used as an antiseptic for many years and it is known that certain concentrations of sodium chloride are effective for inhibiting the growth of various microorganisms (J. D. Reid, Am. J. Hygiene 1932, 16, 540). Without intending to be bound by theory, it is likely that sodium chloride in the composition described herein acts as an antiseptic or antimicrobial and also changes the ionic strength of the composition.

Suitable alkali metal halide salts are available commercially from a range of suppliers. For example, food grade sodium chloride (NaCl) can be used and is widely available.

The amount of alkali metal halide salt in the composition will depend on the specific alkali metal halide salt used and/or the type of water system to be cleaned and/or disinfected. In certain embodiments, the alkali metal halide salt is present in an amount of from about 5% w/v to about 50% w/v such as about 5% w/v, about 6% w/v, about 7% w/v, about 8% w/v, about 9% w/v, about 10% w/v, about 11% w/v, about 12% w/v, about 13% w/v, about 14% w/v, about 15% w/v, about 16% w/v, about 17% w/v, about 18% w/v, about 19% w/v, about 20% w/v, about 21% w/v, about 22% w/v, about 23% w/v, about 24% w/v, about 25% w/v, about 26% w/v, about 27% w/v, about 28% w/v, about 29% w/v, about 30% w/v, about 31% w/v, about 32% w/v, about 33% w/v, about 34% w/v, about 35% w/v, about 36% w/v, about 37% w/v, about 38% w/v, about 39% w/v, about 40% w/v, about 41% w/v, about 42% w/v, about 43% w/v, about 44% w/v, about 45% w/v, about 46% w/v, about 47% w/v, about 48% w/v, about 49% w/v, or about 50% w/v. In certain embodiments, the alkali metal halide salt is sodium chloride and the concentration of sodium chloride in the composition is from about 5% w/v to about 20% w/v, such as about 20% w/v.

The composition also comprises at least one antibacterial compound. In certain embodiments, the antibacterial compound is a bacterial quorum sensing inhibitory compound. The bacterial quorum sensing inhibitory compound may be a natural compound, such as a furanone, for example a 2-(5H)-furanone such as an ascorbate salt. Quorum sensing regulates bacterial virulence phenotypes such as bacterial adhesion and biofilm formation (El-Mowafy et al., Journal of Applied Microbiology 2014 117, 1388-1399). Ascorbic acid provides a degree of protection against adhesion and colonisation by some uropathogens (Habash et al., Can J Microbiol 1999 45, 691-694). In certain embodiments, the antibacterial compound is ascorbic acid and/or a mineral ascorbate. The mineral ascorbate may be selected from the group consisting of sodium ascorbate, calcium ascorbate, potassium ascorbate, and magnesium ascorbate. In certain embodiments, the antibacterial compound is sodium ascorbate. In certain other embodiments, the antibacterial compound is calcium ascorbate. Ascorbic acid (Vitamin C) or sodium ascorbate is a quorum sensing inhibitor with a cytotoxic effect. Quorum sensing inhibitors are able to break the bonds between bacteria and micro-organisms and in a circulating water system results in them un-attaching from surfaces and flowing freely within the pipe work, which can then be flushed from the system.

Suitable antibacterial compounds are available commercially from a range of suppliers.

The amount of antibacterial compound in the composition will depend on the specific antibacterial compound salt used and/or the type of water system to be cleaned and/or disinfected. In certain embodiments, the antibacterial compound is present in an amount of from about 0.5% w/v to about 20% w/v, such as about 0.5% w/v, about 0.6% w/v, about 0.7% w/v, about 0.8% w/v, about 0.9% w/v, about 1% w/v, about 2% w/v, about 3% w/v, about 4% w/v, about 5% w/v, about 6% w/v, about 7% w/v, about 8% w/v, about 9% w/v, about 10% w/v, about 11% w/v, about 12% w/v, about 13% w/v, about 14% w/v, about 15% w/v, about 16% w/v, about 17% w/v, about 18% w/v, about 19% w/v, or about 20% w/v. In certain embodiments, the antibacterial compound is sodium ascorbate and the concentration of sodium ascorbate in the composition is from about 1% w/v to about 5% w/v, such as about 2.5% w/v.

The at least one carboxylic acid, at least one alkali metal halide salt, and at least one antibacterial compound may optionally be combined in either an aqueous or nonaqueous vehicle as desired. Examples of aqueous vehicles that can be used include water. Examples of non-aqueous vehicles that can be used include alcohols, such as ethanol or isopropanol, propylene glycol and polyethylene glycol. The vehicle is preferably a non-toxic and/or a food grade material. The person skilled in the art can readily determine whether an aqueous or non-aqueous vehicle is suitable by assessing the solubility of the components in the vehicle and considering the toxicity and other known properties of the vehicle.

Optionally, one or more additives or functional chemicals can be added to the composition to produce varying effects. Suitable additives or functional chemicals that can be used include dyes, perfumes, biocides, corrosion inhibitors, dispersants, surfactants, reducing agents and pH adjusters.

In certain embodiments, the composition further comprises one or more dyes. The dye may be a food grade dye. A dye can be can be added to the composition to assist in visualising the composition. Food grade dyes are commercially available from suppliers such as Dye Manufacturers of Australia Pty Ltd. For example, brilliant blue FCF can be used as a blue dye or ponceau red 4R can be used as a red dye. The dye may be present in the composition in an amount of from about 0.5% w/v to about 5% w/v, such as about 1% w/v or about 2% w/v.

In certain embodiments, the composition further comprises one or more mineral acids. The mineral acid may be selected from the group consisting of hydrochloric acid, sulfuric acid, amido sulfuric acid, nitric acid, phosphoric acid, hydrofluoric acid, and sulfamic acid.

In certain embodiments, the composition further comprises one or more additional biocides. Additional biocides can be added when microbial contamination is severe. Suitable biocides include oxidizing biocides. Oxidizing biocides include, but are not limited to, chlorine bleach, chlorine, bromine and materials capable of releasing chlorine and bromine. The amount of oxidizing biocide may be from about 0.1 ppm to about 200 ppm, such as from about 5 ppm to about 20 ppm.

In certain embodiments, the composition further comprises one or more corrosion inhibitors. Corrosion inhibitors can be added to reduce corrosion of the metal in a water system. Corrosion inhibitors for multi-metal protection include triazoles, such as, but not limited to, benzotriazole, halogenated triazoles, nitro-substituted azole, and other triazoles as listed in U.S. Pat. No. 5,874,026, which is incorporated by reference in its entirety.

In certain embodiments, the composition further comprises one or more dispersants. Dispersants can be added when needed to keep particulate matter present in the water of a water system dispersed, so that it does not agglomerate and cause fouling during the cleaning and disinfecting process. Dispersants are typically low molecular weight anionic polymers, with “low” referring to a weight average molecular weight of from about 500 to about 20,000. These polymers are typically, but not limited to, acrylic acid, polymaleic acid, copolymers of acrylic acid with sulfonated monomers and alkyl esters thereof. These polymers can include terpolymers of acrylic acid, acrylamide and sulfonated monomers. These polymers can also include quad-polymers consisting of acrylic acid and three other monomers.

In certain embodiments, the composition further comprises one or more surfactants. Surfactants may be added when and where needed to enhance the cleaning and disinfection process. Surfactants useful in water systems include, but are not limited to, ethylene oxide propylene oxide copolymers, linear alkylbenzene sulfonates, ethoxylated phosphate esters, and alkyl polyglycosides, and other surfactants described in U.S. Pat. Nos. 6,139,830, 5,670,055 and 6,080,323, which are all incorporated by reference.

In certain embodiments, the composition further comprises one or more reducing agents. Reducing agents may be added when and where needed to react with oxidants present in order to prepare the water for discharge in compliance with state and local environmental regulations. Suitable reducing agents include, but are not limited to, sodium thiosulfate, sodium bisulfite, sodium metabisulfite and sodium sulfite.

In certain embodiments, the composition further comprises one or more pH adjusters. pH adjusters may be added when needed to adjust the pH of the water being discharged from the industrial water system. Suitable pH adjusting chemicals include, but are not limited to, NaOH, KOH, Ca(OH)₂, Na₂CO₃, and K₂CO₃.

The one or more additives or functional chemicals may be included in the composition or they can be used separately from the composition.

In certain embodiments, the composition is a non-toxic liquid. In certain embodiments, all of the components of the composition are food grade products and, therefore, the composition is classified as a food.

In certain embodiments, the composition is substantially hypochlorite free.

The composition may be packaged in at least one container. For example, the composition may be packaged in a disinfectant hand-pump spray container, a disinfectant pressurised aerosol spray canister or a bottle of liquid disinfectant solution for use in soaking or flushing applications. Following packaging, the composition is ready for transportation to a customer/point of use.

The composition may be made available in a liquid form and dispensed as a solution, an emulsion, a dip, a liquid spray, an aerosol spray, or similar. Alternatively, the composition may be made available as a liquid coating on a substrate such as in the form of a disinfectant handwipe, disinfectant towelette, and the like.

In use, surfaces can be treated by contacting them with the composition. For example, the composition can be poured or sprayed onto a surface or the surface can be dipped into the composition. The composition can also be injected or otherwise introduced into suitable conduits or containers at the point of use, such as (but not limited to) a water treatment, water distribution, and/or water recirculation system.

In use, the composition can be diluted prior to use. In certain embodiments, the composition is diluted in a 1:1 (v/v) ratio with water or other aqueous solution. The dilution ratio (i.e. composition:water) may be from 2:8 to 99:1.

The composition can be used to eliminate or reduce the count of any one or more of a range of Gram-negative bacteria, including (but not limited to) bacteria in the Legionella genus including L. pneumophila, bacteria in the Pseudomonas genus including P. aeruginosa, and/or bacteria in the Salmonella genus including S. bongori and S. enterica. Alternatively, or in addition, the composition can be used to eliminate or reduce the count of any one or more of a range of Gram-positive bacteria in the Streptococcus genus including S. pyogenes, S. agalactiae, S. dysgalactiae, S. bovis, S. anginosus, S. sanguinis, S. mitis, S. mutans, and S. pneumoniae. Alternatively, or in addition, the composition can be used to eliminate or reduce the count of any one or more of a range of amoebae in the Naeglaria genus including Naeglaria fowleri.

The composition can be used to clean and disinfect water supply lines, containers, cooling towers or other surfaces. It will kill Legionella, Pseudomonas, Salmonella, Streptococcus, Naeglaria and others. The composition is designed for but not limited to cooling water systems, industrial water systems, air conditioning systems, evaporative air conditioning systems, warm water systems, potable water systems, beverage dispensary systems, food processing and washing systems (meat, vegetables, sugar, sugar cane, poultry, fruit), food preparation areas, skin of animals and humans, contaminated open wounds and tissue, dermal wound sites and/or lesions of living organisms such as animals and humans.

The inventors/applicants studies have shown that the composition cleans biofilm, scale, and oxidisation from pipes as well as disrupting the bacteria that inhabit biofilms.

Advantageously, an entire water distribution system can be treated with minimal interruption to facility operation, thereby preventing the need for facility shutdown for hazardous acid treatment or pipe removal and replacement.

The composition can be used to clean and/or disinfect fluid conduits (e.g. pipes) and other wetted surfaces of water distribution, water recirculation, and/or water treatment systems. In certain embodiments, a water distribution or water recirculation system may comprise water lines within a building (including, but not limited to, a healthcare facility such as a hospital, a food or beverage processing facility, or an industrial facility). In certain embodiments, a water distribution system may comprise a municipal or community drinking water distribution system arranged to supply potable water to water utilizing facilities of a plurality of different customers. In certain embodiments, a water distribution system may comprise a drinking water distribution system for humans or animals. In certain embodiments, a water distribution system may comprise an agricultural water distribution system. In certain embodiments, a water distribution or water recirculation system may comprise an aquaculture system or hydroponic food production system. In certain embodiments, a water distribution or water recirculation system may comprise a cooling water system, such as may include one or more cooling towers or other heat exchange apparatuses. In certain embodiments, a water treatment or water recirculation system may comprise a wastewater system.

Advantageously, the composition as described herein can be used to eliminate persistent pathogens such as Legionella and/or Pseudomonas bacteria from water distribution systems or any surface. Indeed, independent Pseudomonas challenge tests conducted on behalf of the applicant have demonstrated that the composition as described herein at 100%, 25% and 50% concentrations in water kills all measurable Pseudomonas aeruginosa bacteria in samples tested. P. aeruginosa is a multidrug resistant pathogen that is associated with serious illnesses such as hospital acquired infections and various sepsis syndromes. Therefore, control of P. aeruginosa is vitally important in clinical and aged-care environments.

Advantageously, the aforementioned Pseudomonas challenge tests demonstrated that the composition described herein kills P. aeruginosa on contact. Therefore, the composition may be used as a surface disinfectant for application to a range of surfaces including, but not limited to, skin.

Advantageously, independent tests conducted on behalf of the applicant have also demonstrated that the composition as described herein is effective at removing or otherwise degrading biofilms, with studies showing at least a 1000 fold reduction in the amount of bacteria present in temperate water treated with a 10% (v/v) aqueous solution of the composition, and a complete removal of bacteria in cold water treated with a 10% (v/v) aqueous solution of the composition.

The composition can be used to clean and/or disinfect industrial water systems, such as: cooling water systems, including open recirculating, closed and once-through cooling water systems; boilers and boiler water systems; petroleum wells, downhole formations, geothermal wells and other oil field applications; mineral process waters including mineral washing, flotation and benefaction; paper mill digesters, washers, bleach plants, stock chests, and white water systems, and paper machine surfaces; black liquor evaporators in the pulp industry; gas scrubbers and air washers; continuous casting processes in the metallurgical industry; air conditioning and refrigeration systems; industrial and petroleum process water; indirect contact cooling and heating water, such as pasteurization water; water reclamation systems, water purification systems; membrane filtration water systems; food processing streams (meat, vegetable, sugar beets, sugar cane, grain, poultry, fruit and soybean); and waste treatment systems as well as in clarifiers, liquid-solid applications, municipal sewage treatment, municipal water systems, potable water systems, aquifers, water tanks, sprinkler systems and water heaters.

The composition can be also be used to clean and/or disinfect water system equipment, floors, walls, and drains.

The composition can be also be used to clean and/or disinfect medical devices, medical equipment, and associated surfaces.

Furthermore, the composition may be used to treat skin infections caused by pseudomonas resistant to antibiotics. Thus, the composition may be in the form of a lotion for applying to skin infections or a spray to kill hospital born bacteria that cause significant health issues and in some instances death within hospitals and health related industries.

The present disclosure also provides a method for cleaning and/or disinfecting a surface. The process comprises contacting the surface to be cleaned or disinfected with a composition of the first aspect for a temperature and a time that is sufficient to clean and/or disinfect the surface.

The surface to be cleaned and/or disinfected can be a surface contained in a water supply line, container, cooling tower or similar surface, a medical device, medical equipment and associated surface.

Thus, provided herein is a method of cleaning and/or disinfecting a water system, the method comprising: a) providing a water system; b) adding to the water of the water system a composition of the first aspect; c) allowing the water to circulate through the water system; and d) draining the water from the water system.

The water in the water system may be allowed to circulate for a time period of from about 30 minutes to about 72 hours, such as from about 1 hour to about 24 hours.

If desired, the progress of the cleaning and/or disinfection can be monitored by using standard techniques to determine the amount of inorganic or organic materials present in the water. For instance, it is expected that the amount of inorganic or organic materials present in the water will rise as the cleaning progresses and when the amount of these materials in the water plateaus it indicates that the cleaning is complete.

After the cleaning and/or disinfection is finished, additional mechanical cleaning of the water contact surfaces may be used to remove any accumulation of loose dirt and other undesired material collected in the water system.

Once all of the cleaning and/or disinfection is finished, it is possible to refill the water and begin operation of the water system. For heavily contaminated industrial water systems it is also possible to fill the industrial water system with water and conduct the method again.

The surface to be cleaned and/or disinfected could also be a surface in a hospital or other location where microbial infection is to be minimised or avoided or the skin of an animal to treat, prevent, ameliorate or minimise a microbial infection.

Advantageously, the compositions and methods described herein remove deposits from virtually all wettable surfaces, and disinfects bulk water and all wettable surfaces.

### Example 1—Heterotroph/Biofilm Reduction on Cross Linked Polyethylene Pipe

Samples of tempered water at 43.2° C. (T1) and cold water at 17.2° C. (C1) were taken from WM outlets before the system had been flushed. The system was then flushed with 5 litres of a 1:1 diluted cleaning and/or disinfecting composition containing 9.9% acetic acid, 20% sodium chloride 2.5% sodium ascorbate and blue dye (LGE1) from each outlet. Samples T2 & C2 were taken. The LegioGuard Ultra water system was then powered down and flushing was undertaken post system only with the tanks bypassed. The break tank was dosed with 10% concentration of LGE1 (i.e. 6 litres in 60). The tempered and cold lines were flushed until LGE1 was evident in the samples. Samples T3 & C3 were then taken. After a period of 30 mins, 5 litres was flushed through with 10% concentration of LGE1. Samples T4 & C4 were taken. The break tank was disconnected and both circuits were flushed with mains water until LGE1 was no longer evident. Samples T5 & C5 were taken. A further 5 litres was flushed through the outlets and immediately after samples T6 & C6 were taken.

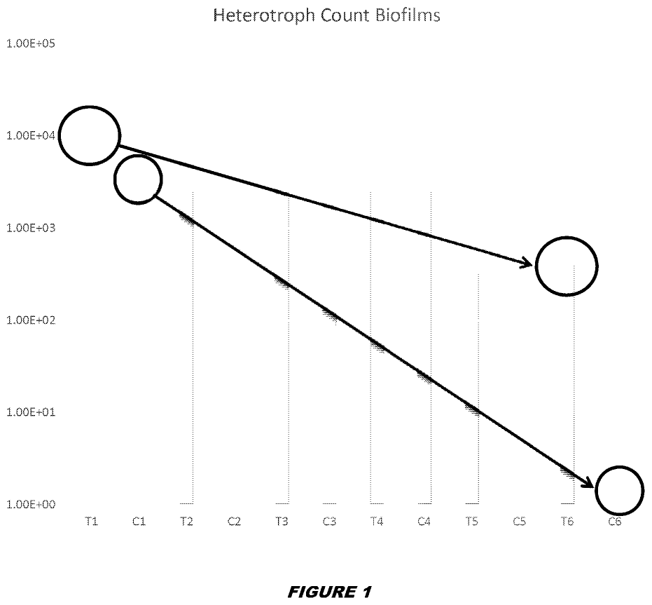

The results are shown in FIG. 1.

### Example 2—Treatment of Legionella Bacteria

Neat and 50% concentration samples of a composition containing 9.9% acetic acid, 20% sodium chloride and 2.5% sodium ascorbate were challenged with Legionella pneumophila serogroup 1 (stock of 22, 00 cfu/ml).

The samples were kept in a shaker and test aliquots were collected at 15 minutes and 30 minutes and analysed as per ALS method MM570 (ISO 11731-2).

Two process control samples (Legionella culture only, without the cleaning and/or disinfectant composition) were set up in parallel and test aliquots were analysed at 15 minute and 30 minute time intervals.

Blank samples (cleaning and/or disinfectant composition only) were also set up as a negative control and treated in parallel with the above samples.

The analysis involved filtering of the test sample aliquots and incubation with appropriate media at 36° C. up to 10 days. The test plates were observed for any visible signs of growth from Day 3 onwards. The presumptive samples (with growth) were confirmed for the spiked organism. The results are shown in Table 1.

TABLE 1

| | Cleaning | Cleaning | Cleaning | Cleaning | | |
|---|---|---|---|---|---|---|
| | and/or | and/or | and/or | and/or | | |
| | disinfectant | disinfectant | disinfectant | disinfectant | | |
| | composition- | composition - | composition - | composition - | Process | Process |
| | Neat at 15 | Neat at 30 | 50% at 15 | 50% at 30 | Control 15 | Control 30 |
| | minutes | minutes | minutes | minutes | minutes | minutes |
| Legionella | <1 | <1 | <1 | <1 | 1500 | 2200 |
| Pneumophila | | | | | | |
| Serogroup 1 | | | | | | |
| (cfu/ml) | | | | | | |

### Example 3—Treatment of Legionella, E. Coli and Pseudomonas Bacteria

25% concentration samples of a composition containing 9.9% acetic acid, 20% sodium chloride and 2.5% sodium ascorbate were challenged with E. coli, Legionella pneumophila serogroup 1, Legionella pneumophila serogroup 2-14, Legionella spp and Pseudomonas aeruginosa using the methods described in Example 2. The results are shown in Table 2.

TABLE 2

| | | | Positive | Negative |
|---|---|---|---|---|
| Analyte | Units | Sample | control | control |
| E. coli, Coliforms - | cfu/100 mL | <10 | 180 | <10 |
| E. coli | | | | |
| Legionella pneumophila | cfu/10 mL | <1 | 320 | <1 |
| serogroup 1 | | | | |
| Legionella pneumophila | cfu/10 mL | <1 | <1 | <1 |
| serogroup 2-14 | | | | |
| Legionella spp | cfu/10 mL | <1 | <1 | <1 |
| Legionella Total | cfu/10 mL | <1 | 320 | <1 |
| Pseudomonas aeruginosa | cfu/100 mL | <10 | 1200 | <10 |

### Example 4—In-Situ Treatment of Water Systems in Commercial Facilities

Water systems in working commercial facilities were treated with a cleaning and/or disinfecting composition solution that was diluted 1 part solution to 3 parts water (25% concentration). The diluted solution contained <2.5% acetic acid, 5% sodium chloride and >0.6% sodium ascorbate, and blue dye.

The water distribution system of the facility being tested was disconnected and simple plumbing connections made to allow the cleaning and/or disinfecting composition solution to be circulated through the warm water loop. 200 L of concentrate was added to 600 L of water in a dosing tank and then circulated through the warm water flow and return system of the facility. The solution was allowed to rest within the distribution system for 30 minutes. The warm water system was then re-instated and flushed with town supply water. All warm water fixtures were then turned on and allowed to run with fresh water for 5-10 minutes or until sediment and scale ceased.

Water samples were taken from water system outlets at various locations in the facility and the individual water samples were subsequently independently analysed and the results from water obtained from one facility are shown in Table 3.

TABLE 3

| | | | Sample | | | |
|---|---|---|---|---|---|---|
| | | | | Before | | After |
| | | | Before | treatment - | After | treatment - |
| | | | treatment - | after 1 minute | treatment - | after 1 minute |
| | | | first flush | flush | first flush | flush |
| | | | Compound | | | |
| | | | EB1826044-005 | EB1826044-006 | EB1829925-001 | EB1829925-002 |
| | LOR | Unit | Result | Result | Result | Result |
| Heterotrophic | 1 | CFU/mL | ~51000 | ~9900 | <1 | <1 |
| Plate Count | | | | | | |
| (36° C.) | | | | | | |
| Legionella | 10 | CFU/mL | <10 | <10 | <10 | <10 |
| pneumophila | | | | | | |
| Legionella | 10 | CFU/mL | 70 | 130 | <10 | <10 |
| Species | | | | | | |
| Total | 10 | CFU/mL | 70 | 130 | <10 | <10 |
| Legionella | | | | | | |

The results show that the heterotrophic plate count decreases over time after flushing with the composition and that the cleaning and/or disinfecting composition removes any detectable amounts of Legionella bacteria and heterotroph/biofilm. Similar results were obtained from other facilities.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

It will be appreciated by those skilled in the art that the invention is not restricted in its use to the particular application described. Neither is the present invention restricted in its preferred embodiment with regard to the particular elements and/or features described or depicted herein. It will be appreciated that the invention is not limited to the embodiment or embodiments disclosed, but is capable of numerous rearrangements, modifications and substitutions without departing from the scope of the invention as set forth and defined by the following claims.

1. A cleaning and/or disinfecting composition suitable for cleaning and/or disinfecting fluid conduits and other wetted surfaces of water distribution, water recirculation, and/or water treatment systems, the composition comprising: from about 5% w/v to about 50% w/v of at least one carboxylic acid; from about 5% w/v to about 50% w/v of at least one alkali metal halide salt; and from about 0.5% w/v to about 20% w/v of at least one antibacterial compound.

2. The cleaning and/or disinfecting composition of claim 1, wherein the carboxylic acid is a C₁ to C₆ carboxylic acid.

3. The cleaning and/or disinfecting composition of claim 1, wherein the carboxylic acid is acetic acid.

4. The cleaning and/or disinfecting composition of claim 3, wherein the acetic acid is present in the composition in an amount of from about 5% w/v to about 15% w/v.

5. The cleaning and/or disinfecting composition of claim 3, wherein the acetic acid is present in the composition in an amount of about 9.9%.

6. The cleaning and/or disinfecting composition of claim 1, wherein the alkali metal halide salt is sodium chloride.

7. The cleaning and/or disinfecting composition of claim 6, wherein the sodium chloride is present in the composition in an amount of about 20% w/v.

8. The cleaning and/or disinfecting composition of claim 1, wherein the antibacterial compound is sodium ascorbate.

9. The cleaning and/or disinfecting composition of claim 8, wherein the sodium ascorbate is present in the composition in an amount of about 2.5% w/v.

10. The cleaning and/or disinfecting composition claim 1, wherein the composition eliminates or reduces the count of any one or more of: Gram-negative bacteria, including bacteria in the Legionella genus including L. pneumophila, bacteria in the Pseudomonas genus including P. aeruginosa, and/or bacteria in the Salmonella genus including S. bongori and S. enterica; Gram-positive bacteria in the Streptococcus genus including S. pyogenes, S. agalactiae, S. dysgalactiae, S. bovis, S. anginosus, S. sanguinis, S. mitis, S. mutans, and S. pneumoniae; and/or amoebae in the Naeglaria genus including Naeglaria fowleri.

11. A method for cleaning and/or disinfecting a surface, the process comprising contacting the surface to be cleaned or disinfected with the composition of claim 1 for a temperature and a time that is sufficient to clean and/or disinfect the surface.

12. A method of cleaning and/or disinfecting a water system comprising the steps of: a) providing a water system; b) adding to the water of the water system the composition of claim 1; c) allowing the water to circulate through the water system; and d) draining the water from the water system.
